(19) United States
(12) Patent Application Publication
(10) Pub. No.: US 2003/0162197 A1
(43) Pub. Date: Aug. 28, 2003

(54) Method of detecting neoplastic or non-neoplastic cells

(76) Inventors: Alexander Alan Morley, South Australia (AU); Pamela Joy Sykes, South Australia (AU); Michael Julian Brisco, South Australia (AU)

Correspondence Address: MORRISON & FOERSTER LLP, 1650 TYSONS BOULEVARD, SUITE 300, MCLEAN, VA, 22102, US

(21) Appl. No.: 10/257,445

(22) PCT Filed: Apr. 12, 2001
(86) PCT No.: PCT/AU01/00429

(30) Foreign Application Priority Data
Apr. 13, 2000 (AU) PQ6876

(51) Int. Cl. C12Q 1/68

(52) U.S. Cl. 435/6

(57) ABSTRACT

The present invention relates to a method of qualitatively and/or quantitatively monitoring the progress of a lymphoid neoplasia in a mammal and, more particularly, to a method of monitoring the progress of a lymphoid neoplasia utilising a molecular screening technique. The method of the present invention is useful in a range of applications including, but not limited to, monitoring the progression of a neoplastic lymphoid disease condition, monitoring the levels of neoplastic lymphoid cells during remission, predicting the likelihood of a subject's relapse from a remissive state to disease state or for assessing the effectiveness of existing therapeutic drugs and/or new therapeutic agents. In a related aspect, the present invention also provides a method for qualitatively and/or quantitatively detecting clonal lymphocyte populations in a mammal and, more particularly, for detecting multiple clonal lymphocyte populations in a subject such as where a neoplastic lymphoid cell has undergone somatic gene rearrangement thereby forming a new and genetically distinct clonal population or such as where one or several clonal lymphocyte populations contribute to an immunological response.

### FIELD OF THE INVENTION

The present invention relates to a method of qualitatively and/or quantitatively monitoring the progress of a lymphoid neoplasia in a mammal and, more particularly, to a method of monitoring the progress of a lymphoid neoplasia utilising a molecular screening technique. The method of the present invention is useful in a range of applications including, but not limited to, monitoring the progression of a neoplastic lymphoid disease conditions monitoring the levels of neoplastic lymphoid cells during remission, predicting the likelihood of a subject's relapse from a remissive state to disease state or for assessing the effectiveness of existing therapeutic drugs and/or new therapeutic agents. In a related aspect, the present invention also provides a method for qualitatively and/or quantitatively detecting clonal lymphocyte populations in a mammal and, more particularly, for detecting multiple clonal lymphocyte populations in a subject such as where a neoplastic lymphoid cell has undergone somatic gene rearrangement thereby forming a new and genetically distinct clonal population or such as where one or several clonal lymphocyte populations contribute to an immunological response.

### BACKGROUND OF THE INVENTION

The reference to any prior art in this specification is not, and should not be taken as, an acknowledgment or any form of suggestion that that prior art forms part of the common general knowledge in Australia.

Lymphocytes, the cells that subserve the immune response, are of two types—B lymphocytes which produce antibody and T lymphocytes which are involved in cellular immunity. During development, in order to develop a specific immune response, each lymphocyte rearranges in a unique fashion one or a few specific genes—the immunoglobulin genes for a B lymphocyte and the T cell receptor genes for a T lymphocyte. All descendants of these lymphocytes will then carry the same rearrangement.

A neoplasm is believed to arise as the result of summation of genetic changes in a single cell. Subsequent proliferation of that cell gives rise to a population of descendants. Neoplasms arising from malignant change in a B or T lymphocyte (often referred to as “cancers”) would therefore predominantly comprise a clone of cells, each of which contains the same gene rearrangements that were present in the founder cell. Secondary gene rearrangements may occur within the neoplastic clone leading to genetically different subclones.

Lymphocytic neoplasms are therefore clonal disorders. They develop after one or more mutations in a single cell cause the cell and its progeny to multiply progressively and exponentially. For example, when lymphocytic leukemic clones number 10¹¹ to 10¹² cells in the body, clinical symptoms ensue. Without treatment, the clone continues to expand, and death results when there are approximately 10¹³ leukemic cells. If, however, the patient receives cytotoxic treatment, the clone decreases in size, and it can no longer be identified by conventional techniques when it comprises fewer than about 10¹⁰ cells. At this point, the patient is judged to be in clinical and haematological remission, although the term “remission”, in fact, refers only to a somewhat arbitrary point toward one end of a continuum of leukemic-cell number. Since the number of leukemic cells that may remain during remission is unknown and may range from 0 to 10¹⁰, treatment after remission has been achieved is empirical, and its intensity is based on various clinical or laboratory prognostic factors determined at diagnosis or early in treatment. Consequently, some patients may receive too little treatment and others may receive too much.

Current methods for quantification of malignant lymphocytes involve the use of a “marker” which is shared by all cells of the clone. The marker may be a surface antigen, or patterns of several surface antigens, or it may be a molecular change. The molecular changes which are used may be broadly classified into two types—those which involve a chromosomal translocation or inversion, and those which will use the immunoglobulin or T cell receptor gene arrangements.

Current methods vary in their complexity, ease of performance, sensitivity and applicability. In general there is a direct relationship between complexity and sensitivity and the most sensitive methods are usually very complex and time consuming. Owing to their complexity, current methods which use gene rearrangements as molecular markers in order to measure the number of neoplastic lymphoid cells are still only suitable for use as research tools and are not suited for widespread clinical use.

With respect to detecting and quantifying minimal residual disease, the essential problem is one of detecting and quantifying a particular marker from a neoplastic lymphoid clone against a background of heterogeneous markers derived from heterogeneous, normal polyclonal lymphocytes. Most approaches to increasing the level of detection have been based upon improving methods to directly and positively detect the neoplastic lymphoid marker itself. For example, PCR based methods have used either probing using a labelled sequence-specific probe, or have used PCR priming using PCR primers specific for the neoplastic lymphoid sequence. A different approach relies upon direct detection of the neoplastic lymphoid phenotype.

An important drawback associated with most currently used molecular techniques which are based on probing or amplifying the DNA of interest, however, is the pre-requisite for nucleotide sequence information in order to design and synthesize suitably specific probes or primers. This necessarily renders such techniques both complex and expensive.

Accordingly, there is a need to develop improved methods for qualitatively and/or quantitatively detecting the levels of neoplastic lymphoid cells in a subject, which methods are highly sensitive yet simple to perform. In work leading up to the present invention, the inventors have developed a simple yet sensitive method for monitoring the progress of a lymphoid neoplasia in a mammal. The simplicity and sensitivity of the method stems from the fact that the inventors have developed a method of detection based on the screening of a biological sample at the molecular level for the presence of a neoplastic lymphocyte specific marker, in particular the rearranged nucleic acid molecule encoding for the T cell receptor or immunoglobulin region, without the need to firstly obtain nucleotide sequence information relating to the subject marker. Nevertheless, the method is highly sensitive.

The inventors have determined that by reducing non-marker background DNA rather than by attempting to specifically probe or to specifically amplify the DNA of interest from a heterogeneous DNA population, a highly sensitive yet simple method of screening for neoplastic cells is made possible. In particular, the inventors have determined that by hybridising the patient test DNA with a sample of the neoplastic lymphocyte DNA obtained at diagnosis, unhybridised test DNA or mismatched hybridisations can be efficiently removed thereby effectively enriching the neoplastic DNA marker population. Detection of the neoplastic signal is then performed against a background of reduced levels of non-marker background DNA. Detection involves a generic detection system such as PCR, fluorescent detection, immunodetection or enzymatic detection. Removal of non-neoplastic background molecules increases the sensitivity of detection irrespective of the detection system which is used.

This method obviates the usual requirement for nucleotide sequence information in order to utilise a molecular screening technique thereby significantly simplifying the performance of the technique while maintaining and in some cases improving upon the upper levels of sensitivity currently available for such techniques.

In a related aspect, based on the specific molecular rearrangement of the T cell receptor or immunoglobulin variable gene in a neoplastic lymphocyte, the present inventors have also adapted the technique of two dimensional gel electrophoresis to provide an efficient and sensitive method for the diagnosis of lymphocytic neoplasia, and in particular, the diagnosis of somatic neoplastic lymphocyte rearrangements which become evident after initial diagnosis (i.e. clonal evolution).

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention provides a method for monitoring a lymphoid neoplastic condition in a mammal, said method comprising contacting the nucleic acid molecules contained in a sample derived from said mammal with a nucleic acid reference molecule or derivative or analogue thereof which is complementary to a marker nucleic acid molecule or analogue thereof, which marker molecule is characteristic of the neoplastic cells, for a time and under conditions sufficient to facilitate the interaction of said reference molecule with said marker molecule, enriching for said marker molecule by reducing the concentration of un-hybridised nucleic acid molecules and hybridisation-mismatched nucleic acid molecules and qualitatively and/or quantitatively detecting said enriched marker nucleic acid molecules.

Another aspect of the present invention provides a method for monitoring a neoplastic lymphoid condition in a mammal, said method comprising contacting the nucleic acid molecules contained in a sample derived from said mammal with a nucleic acid reference molecule or derivative or analogue thereof which is complementary to a portion of a rearranged TCR or immunoglobulin variable region nucleic acid molecule or derivative or analogue thereof for a time and under conditions sufficient to facilitate the interaction of said reference molecule with said variable region nucleic acid molecule, enriching for said variable region nucleic acid molecule by reducing the concentration of unhybridised nucleic acid molecules and hybridisation-mismatched nucleic acid molecules and qualitatively and/or quantitatively detecting said enriched variable region nucleic acid molecules.

Still another aspect of the present invention is directed to a method for monitoring a lymphoid malignant condition in a mammal, said method comprising contacting the nucleic acid molecules contained in a test sample derived from said mammal with a nucleic acid reference molecule or derivative or analogue thereof which is complementary to a rearranged TCR or immunoglobulin variable region nucleic acid molecule or derivative or analogue thereof for a time and under conditions sufficient to facilitate the interaction of said reference molecule with said variable region nucleic acid molecule, enriching for said variable region nucleic acid molecule by reducing the concentration of unhybridised nucleic acid molecules and hybridisation-mismatched nucleic acid molecules and qualitatively and/or quantitatively detecting said enriched variable region nucleic acid molecule.

Yet another aspect of the present invention is directed to a method for monitoring a lymphoid malignant condition in a human said method comprising contacting the nucleic acid molecules contained in a test sample derived from said human with a nucleic acid reference molecule or derivative or analogue thereof which is complementary to a rearranged TCR or immunoglobulin variable region nucleic acid molecule or derivative or analogue thereof for a time and under conditions sufficient to facilitate the interaction of said reference molecule with said variable region nucleic acid molecule, enriching for said variable region nucleic acid molecule by reducing the concentration of unhybridised nucleic acid molecules and hybridisation-mismatched nucleic acid molecules and qualitatively and/or quantitatively detecting said enriched variable region nucleic acid molecule.

Still yet another aspect of the present invention provides a method for monitoring a neoplastic lymphoid condition in a mammal, said method comprising contacting the nucleic acid molecules contained in a test sample derived from said mammal with a nucleic acid driver molecule or derivative or analogue thereof which is complementary to a rearranged TCR or immunoglobulin variable region nucleic acid molecule or derivative or analogue thereof for a time and under conditions sufficient to facilitate the interaction of said driver molecule with said variable region nucleic acid molecule, enriching for said variable region nucleic acid molecule by reducing the concentration of unhybridised nucleic acid molecules, hybridisation-mismatched nucleic acid molecules and nucleic acid driver molecules and qualitatively and/or quantitatively detecting said enriched variable region nucleic acid molecule.

A further aspect of the present invention is directed to a method for detecting and/or quantifying a clonal population of cells in a biological sample said cells being characterised by a marker nucleic acid molecule, which marker nucleic acid molecule is electrophoretically co-migratable within said population of cells, said method comprising electrophoretically separating the nucleic acid molecules contained in said sample, wherein said separation is based on nucleic acid length and sequence, and detecting said separated nucleic acid molecules.

Another further aspect of the present invention more particularly provides a method for detecting and/or quantifying a population of neoplastic lymphoid cells in a biological sample said samples being characterised by a marker nucleic acid molecule, which marker nucleic acid molecule is electrophoretically co-migratable within said population of cells, said method comprising electrophoretically separating nucleic acid molecules contained in said sample, wherein said separation is based on nucleic acid length and sequence, and detecting said separated nucleic acid molecules.

Yet another further aspect of the present invention provides a method for detecting and/or quantifying a population of neoplastic lymphoid cells in a biological sample said neoplastic cells being characterised by a marker nucleic acid molecule, which marker nucleic acid molecule is electrophoretically co-migratable within said population of cells, said method comprising electrophoretically separating the nucleic acid molecules contained in said sample, wherein said separation is two dimensional denaturing gradient gel electrophoresis and is based on nucleic acid length and sequence, and detecting said separated nucleic acid molecules.

Still yet another further aspect of the present intention provides a method for detecting and/or quantifying neoplastic cells in a mammal, said neoplastic cells being characterised by a marker nucleic acid molecule which marker nucleic acid molecule is electrophoretically co-migratable within said population of cells, said method comprising electrophoretically separating nucleic acid molecules contained in a sample derived from said mammal, wherein said separation is based on nucleic acid length and sequence, and detecting said separated nucleic acid molecules.

In another aspect there is provided a method for detecting and/or quantifying multiple non-neoplastic lymphoid cells in a biological sample, said non-neoplastic cells being characterised by a marker nucleic acid molecule, which marker nucleic acid molecule is electrophoretically co-migratable within said population of cell, said method comprising electrophoretically separating the nucleic acid molecules contained in said sample, wherein said separation is based on nucleic acid length and sequence, and detecting said separated nucleic acid molecules.

Preferably said separation is two dimensional denaturing gradient gel eletrophoresis.

### BRIEF DESCRIPTION OF THE DRAWINGS

FIG. 1 is a graphical representation of two experiments in which different amounts of unknown marker leukaemic DNA were added to normal DNA and the amounts of the unknown were measured by the method described in Example 2.

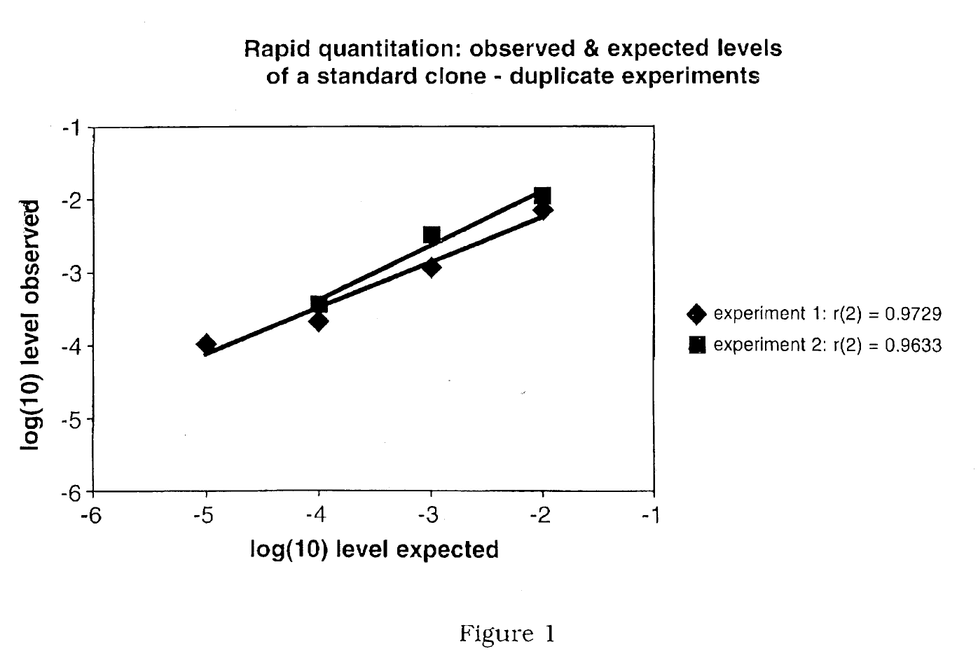

FIG. 2 is an image of he detection of a cell line DNA mixed in various proportions with DNA from peripheral blood mononuclear cells, by identification of the genetic marker. CDR3 regions of the Immunoglobulin heavy chain gene act as a clone-specific marker. DNA from a B-cell line was mixed with DNA extracted from peripheral blood in various proportions, to give ratios of cell line cells: blood cells from 1:3 to 1:3000. CDR3 regions were amplified by PCR, using GC clamped primers. Products were electrophoresed on a 6% polyacrylamide gel. Arrows indicate two marker products from the cell line, of 130 and 120 base pairs, whose size is specific for the cell line. These can be seen in the 1 in 3, 1 in 10, and 1 in 30 mixes, but not in higher dilutions.

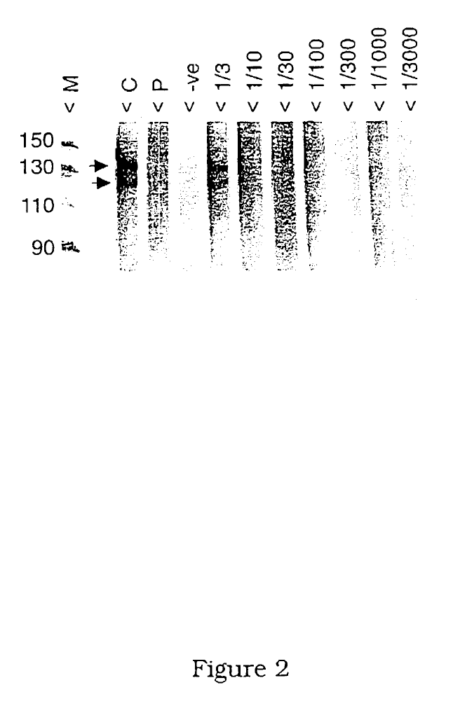

| | |
|---|---|
| M: | markers, with sizes (base pairs) to the left; |
| C: | cell line DNA alone |
| P: | peripheral blood cells DNA alone |
| -ve: | negative control (no DNA) |
| Other tracks: | cell line cells per blood cell. |

FIG. 3 is an image of the products detailed in FIG. 2, above, analysed by 2 dimensional denaturing gradient gel electrophoresis. The above products were analysed first by electrophoresis on a standard polyacrylamide gel. The strip of gel containing the products was cut out, and placed on the top of a denaturing gradient gel, containing a concentration gradient of urea and formamide, from 0% saturation at the top, to 80% saturation at the bottom, according to standard recipes. Products were electrophoresed overnight.

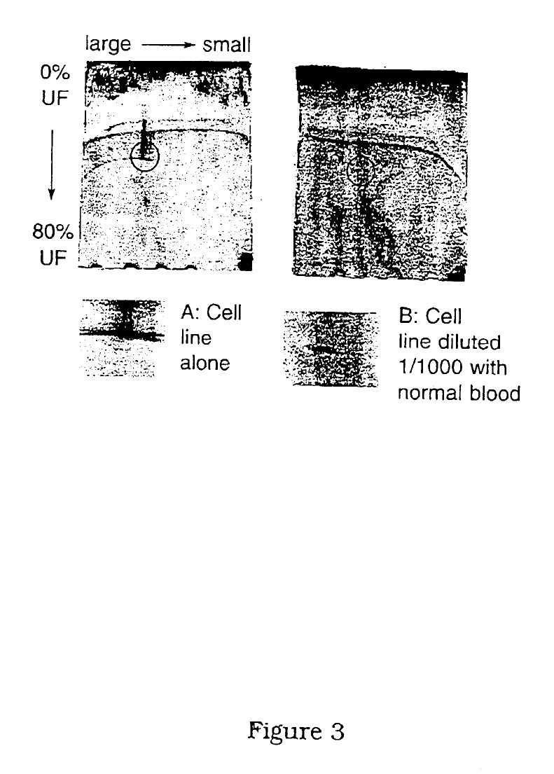

→ direction of 1^{st} dimension separation: by size, on a standard 6% polyacrylamide gel;

↓ direction of 2^{nd} dimension separation: by melting characteristics, on a concentration gradient of denaturant (urea-formamide) in a polyacrylamide gel

A: Cell line alone gives one major product (circled)

B: Cell line diluted shows on high resolution a product (circled) in the corresponding area of gel as the cell line product, and this similar in size and melting characteristics.

Cell line DNA diluted 1/3 to 1/300 also gave this product, but neither blood DNA alone, nor the negative control (no DNA), gave this product In conclusion, two dimensional separation produced approximately 30-fold improvement compared with one dimensional separation.

### DETAILED DESCRIPTION OF THE INVENTION

The present invention is predicated, in part, on the use of a reference nucleic acid molecule to both identify a neoplastic marker nucleic acid molecule and to provide a means for reducing non-marker background nucleic acid molecules which are present in the test sample. This has facilitated the development of a simple yet highly sensitive method for monitoring a neoplastic lymphoid condition. In a related aspect, the inventors have developed a technique for identifying and/or monitoring populations of clonal cells, such as neoplastic cells, based on the co-migration patterns of marker nucleic acid molecules through a 2 dimensional electrophoretic separation based on nucleic acid length and sequence.

Accordingly, one aspect of the present invention provides a method for monitoring a neoplastic lymphoid condition in a mammal, said method comprising contacting the nucleic acid molecules contained in a sample derived from said mammal with a nucleic acid reference molecule or derivative or analogue thereof which is complementary to a marker nucleic acid molecule or analogue thereof, which marker molecule is characteristic of the neoplastic cells, for a time and under conditions sufficient to facilitate the interaction of said reference molecule with said marker molecule, enriching for said marker molecule by reducing the concentration of un-hybridised nucleic acid molecules and hybridisation-mismatched nucleic acid molecules and qualitatively and/or quantitatively detecting said enriched marker nucleic acid molecules.

Reference to a “marker nucleic acid molecule” which is “characteristic of” the subject neoplastic lymphoid cell should be understood as a reference to a molecule which is found in a neoplastic lymphoid cell but which is either not found in non-neoplastic cells or which is not found in significant numbers of non-neoplastic cells. By “significant” is meant that detection of the subject marker nevertheless provides a useful indication of the levels of neoplastic lymphoid cells which are present in a subject mammal. The marker may be a nucleic acid molecule, or region thereof, which encodes a proteinaceous molecule such as an intracellular, secreted or transmembrane molecule. Alternatively, it may comprise a non-coding sequence which is nevertheless characteristic of the subject neoplastic cells. The marker molecule may be DNA or RNA, such as mRNA.

Where the marker molecule is a DNA molecule which encodes a proteinaceous molecule, its expression may be constitutive or it may require that a stimulatory signal be received by the neoplastic cell in order to induce its transcription and translation. Since the method of the present invention is directed to screening for the marker nucleic acid molecule per se, where genomic DNA is the subject of detection it is not material whether the marker is expressed or not. However, if the subject method is directed to detecting mRNA, and the protein encoded by said marker is not constitutively produced, it will be necessary to suitably stimulate the subject neoplastic cell prior to screening. Such stimulation may be performed either in vitro after the biological sample comprising the subject neoplastic cells has been harvested from the mammal or a stimulatory signal may be administered to the mammal prior to harvesting of the biological sample. Still further, the marker nucleic acid molecule may be one which is normally found in the subject neoplastic cell prior to its transformation. Alternatively, the marker may be one which is introduced to the subject neoplastic cell at the time of its transformation. For example, where transformation is induced by viral infection of a non-neoplastic cell, the subject marker may be a virus derived or virus specific molecule.

Preferably, the marker is the rearranged genomic variable region nucleic acid molecule or derivative thereof of a T cell receptor (herein referred to as “TCR”) chain or an immunoglobulin chain. Without limiting the present invention in any way, each lymphoid cell undergoes somatic recombination of its germ line variable region gene segments (either V and J or V, D and J segments) depending on the particular gene rearranged in order to generate a total antigen diversity of approximately 10¹⁶ distinct variable region structures. In any given lymphoid cell, such as a T cell or B cell, at least two distinct variable region gene segment rearrangements are likely to occur due to the rearrangement of two or more of the two chains comprising the TCR or immunoglobulin molecule. Specifically, the α, β, γ or δ chains of the TCR and/or the heavy and light chains of the immunoglobulin molecule. In addition to rearrangements of the VJ or VDJ segment of any given immunoglobulin or TCR gene, nucleotides are randomly removed and/or inserted at the junction between the segments. This leads to the generation of enormous diversity.

The present invention therefore more particularly provides a method for monitoring a neoplastic lymphoid condition in a mammal, said method comprising contacting the nucleic acid molecules contained in a sample derived from said mammal with a nucleic acid reference molecule or derivative or analogue thereof which is complementary to a rearranged TCR or immunoglobulin variable region nucleic acid molecule or derivative or analogue thereof for a time and under conditions sufficient to facilitate the interaction of said reference molecule with said variable region nucleic acid molecule, enriching for said variable region nucleic acid molecule by reducing the concentration of unhybridised nucleic acid molecules and hybridisation-mismatched nucleic acid molecules and qualitatively and/or quantitatively detecting said enriched variable region nucleic acid molecule.

Preferably, said variable region nucleic acid molecule is the genomic form of the rearranged variable region gene segment.

It should be understood that reference to “lymphoid cell” is a reference to any cell which has rearranged at least one germ line set of immunoglobulin or TCR variable region gene segments. The immunoglobulin variable region encoding genomic DNA which may be rearranged includes the variable regions associated with the heavy chain or the κ or λ light chain while the TCR chain variable region encoding genomic DNA which may be rearranged include the α, β, γ and δ chains. In this regard, a cell should be understood to fall within the scope of the “lymphoid cell” definition provided the cell has rearranged the variable region encoding DNA of at least one immunoglobulin or TCR gene segment region. It is not necessary that the cell is also transcribing and translating the rearranged DNA. In this regard, “lymphoid cell” includes within its scope, but is in no way limited to, immature T and B cells which have rearranged the TCR or immunoglobulin variable region gene segments but which are not yet expressing the rearranged chain (such as TCR thymocytes) or which have not yet rearranged both chains of their TCR or immunoglobulin variable region gene segments. This definition further extends to lymphoid-like cells which have undergone at least some TCR or immunoglobulin variable region rearrangement but which cell may not otherwise exhibit all the phenotypic or functional characteristics traditionally associated with a mature T cell or B cell. Accordingly, the method of the present invention can be used to monitor neoplasias of cells including, but not limited to, lymphoid cells at any differentiative stage of development, activated lymphoid cells or non-lymphoid/lymphoid-like cells provided that rearrangement of at least part of one variable region gene region has occurred.

It should also be understood that although it is preferable that the rearrangement of at least one variable region gene region has been completed, the method of the present invention is nevertheless applicable to monitoring neoplastic cells which exhibit only partial rearrangement. For example, a B cell which has only undergone the DJ recombination event is a cell which has undergone only partial rearrangement. Complete rearrangement will not be achieved until the DJ recombination segment has further recombined with a V segment. The method of the present invention can therefore be designed to detect the partial or complete variable region rearrangement of one TCR or immunoglobulin chain utilising a reference molecule complementary to this marker sequence or, for example, if greater specificity is required and the neoplastic cell has rearranged the variable region of both TCR or immunoglobulin chains, reference molecules directed to both forms of rearrangement can be utilised.

Reference to a “neoplastic cell” should be understood as a reference to a cell exhibiting abnormal “growth”. The term “growth” should be understood in its broadest sense and includes reference to proliferation. In this regard, an example of abnormal cell growth is the uncontrolled proliferation of a cell. The uncontrolled proliferation of a lymphoid cell may lead to a population of cells which take the form of either a solid tumour or a single cell suspension (such as is observed, for example, in the blood of a leukemic patient). A neoplastic cell may be a benign cell or a malignant cell. In a preferred embodiment, the neoplastic cell is a malignant cell. In this regard, reference to a “neoplastic condition” is a reference to the existence of neoplastic cells in the subject mammal. Although “neoplastic lymphoid condition” includes reference to disease conditions which are characterised by reference to the presence of abnormally high numbers of neoplastic cells such as occurs in leukemias, lymphomas and myclomas, this phrase should also be understood to include reference to the circumstance where the number of neoplastic cells found in a mammal falls below the threshold which is usually regarded as demarcating the shift of a mammal from an evident disease state to a remission state or vice versa (the cell number which is present during remission is often referred to as the “minimal residual disease”). Still further, even where the number of neoplastic cells present in a mammal falls below the threshold detectable by the screening methods utilised prior to the advent of the present invention, the mammal is nevertheless regarded as exhibiting a “neoplastic condition”.

In a preferred embodiment, the present invention is directed to a method for monitoring a lymphoid malignant condition in a mammal, said method comprising contacting the nucleic acid molecules contained in a test sample derived from said mammal with a nucleic acid reference molecule or derivative or analogue thereof which is complementary to a rearranged TCR or immunoglobulin variable region nucleic acid molecule or derivative or analogue thereof for a time and under conditions sufficient to facilitate the interaction of said reference molecule with said variable region nucleic acid molecule, enriching for said variable region nucleic acid molecule by reducing the concentration of unhybridised nucleic acid molecules and hybridisation-mismatched nucleic acid molecules and qualitatively and/or quantitatively detecting said enriched variable region nucleic acid molecule.

Still more preferably, said monitoring is monitoring of the minimal residual disease condition.

Reference to a “test sample” derived from the subject mammal should be understood in its broadest sense to include any sample of material derived from a mammal. This includes reference to both samples which are naturally present in the body of the mammal, such as tissue and body fluids (for example biopsy specimens such as lymphoid specimens, blood, lymph fluid, faeces or bronchial secretions) and samples which are introduced into the body of the mammal and subsequently removed, such as, for example, the saline solution extracted from the lung following a lung lavage or from the colon following an enema. The biological sample which is tested according to the method of the present invention may be tested directly or may require some form of treatment prior to testing. For example, a biopsy sample may require homogenisation prior to testing. Where the sample comprises cellular material, it may be necessary to extract or otherwise expose the nucleic acid material present in the cellular material in order to facilitate interaction of the nucleic acid material with the reference nucleic acid molecule. As detailed earlier, the sample may also require some form of stimulation prior to testing if the test is designed to detect a mRNA marker sequence.

The choice of what type of sample is most suitable for testing in accordance with the method disclosed herein will be dependent on the nature of the neoplastic condition which is being monitored. For example, if the neoplastic condition is a lymphoid leukaemia, a blood sample, lymph fluid sample or bone marrow aspirate would likely provide a suitable testing sample. Where the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one mammal. Choosing an appropriate sample for any given monitoring scenario would fall within the skills of the person of ordinary skill in the art.

The term “mammal” as used herein includes humans, primates, livestock animals (e.g. horses, cattle, sheep, pigs, donkeys), laboratory test animals (e.g. mice, rats, rabbits, guinea pigs), companion animals (e.g. dogs, cats) and captive wild animals (e.g. kangaroos, deer, foxes). Preferably, the mammal is a human or a laboratory test animal. Even more preferably the mammal is a human.

According to this preferred embodiment, the present invention is directed to a method for monitoring a lymphoid malignant condition in a human said method comprising contacting the nucleic acid molecules contained in a test sample derived from said human with a nucleic acid reference molecule or derivative or analogue thereof which is complementary to a rearranged TCR or immunoglobulin variable region nucleic acid molecule or derivative or analogue thereof for a time and under conditions sufficient to facilitate the interaction of said reference molecule with said variable region nucleic acid molecule, enriching for said variable region nucleic acid molecule by reducing the concentration of unhybridised nucleic acid molecules and hybridisation-mismatched nucleic acid molecules and qualitatively and/or quantitatively detecting said enriched variable region nucleic acid molecule.

The method of the present invention is predicated on the use of a reference nucleic acid molecule which can interact with a marker nucleic acid molecule as hereinbefore defined for the purpose of monitoring an actual or presumptive neoplastic lymphoid condition. In this regard, reference to “monitoring” should be understood as a reference to testing a subject for the presence or level of the subject neoplastic lymphoid cells after initial diagnosis of the neoplastic condition. “Monitoring” includes reference to conducting both isolated one off tests or a series of tests over a period of days, weeks, months or years. The tests may be conducted for any of a number of reasons including, but not limited to, predicting the likelihood that a mammal which is in remission will relapse, monitoring the effectiveness of a treatment protocol, checking the status of a patient who is in remission monitoring the progress of the neoplastic condition prior to or subsequent to the application of a treatment regime, in order to assist in reaching a decision with respect to suitable treatment or in order to test new forms of treatment. The method of the present invention is therefore useful as both a clinical tool and as a research tool.

Reference to a “nucleic acid” should be understood as a reference to both a deoxyribonucleic acid and ribonucleic acid or derivatives or analogues thereof.

The reference nucleic acid molecule may be naturally, recombinant or synthetically produced. Where the sequence information relating to the subject marker molecule is known, the reference molecule may be synthetically or recombinantly generated. In a preferred embodiment, however, the present invention is applied to situations where it is not possible and/or not desirable to obtain nucleotide sequence information with respect to the marker molecules. This is of particular relevance where the method of the present invention is applied in a clinical setting to monitoring the neoplastic lymphoid conditions for numerous patients. In particular, since the TCR or immunoglobulin variable region rearrangement is a particularly suitable marker of neoplastic lymphoid cells, it is most likely, that the particular variable region rearrangement exhibited by the neoplastic lymphoid cells of a given patient will be unique. Even where one patient presents twice with primary neoplastic lymphoid conditions, it is likely that variable region rearrangement of the neoplastic cell derived from the first primary neoplastic lymphoid condition will be different to that of the neoplastic lymphoid cell derived from the second primary lymphoid condition.

In a preferred embodiment, the reference nucleic acid molecule is a naturally occurring molecule such as a driver nucleic acid molecule. A “driver” nucleic acid molecule should be understood to refer to a reference nucleic acid molecule which has been isolated from the mammal which is being monitored via the method of the present invention. The molecule is necessarily isolated prior to initially performing the method of the present invention. Although methods of locating, identifying and isolating driver molecules suitable for use in the present invention would be well known to those skilled in the art, in one preferred embodiment the driver molecule is the rearranged TCR or immunoglobulin variable region genomic DNA which has been derived from the sample of neoplastic lymphoid cells obtained from the subject mammal at the time of diagnosis. Driver molecule suitable for use in the method of the present invention may be prepared, for example, by nucleic acid amplification of the re-arranged TCR or immunoglobulin gene present in DNA or RNA extracted from cells obtained at diagnosis. Without limiting the operation of the present invention in any way, diagnosis of a neoplastic lymphoid condition usually occurs as a result of a patient having presented with abnormal symptoms.

These symptoms have usually been brought on by the presence of high concentrations of the neoplastic lymphoid cells (usually greater than 99% in the bone marrow for neoplasias such as B cell leukemias, greater than 95% in the thymus for thymocyte leukemias and greater than 99% of cells comprising solid lymphoid tumours). Accordingly, samples of neoplastic cells can be easily and quickly isolated either at the time of diagnosis or at any time prior to a treatment regime commencing. Sufficient cells or DNA or RNA or driver nucleic acid molecule samples can be obtained and stored (for example as frozen aliquots) from any given patient such that monitoring of the neoplastic lymphoid condition of that patient can be maintained for as long as is required or desired.

The method of the present invention therefore preferably provides a method for monitoring a neoplastic lymphoid condition in a mammal, said method comprising contacting the nucleic acid molecules contained in the test sample derived from said mammal with a nucleic acid driver molecule or derivative or analogue thereof which is complementary to a rearranged TCR or immunoglobulin variable region nucleic acid molecule or derivative or analogue thereof for a time and under conditions sufficient to facilitate the interaction of said driver molecule with said variable region nucleic acid molecule, enriching for said variable region nucleic acid molecules by reducing the concentration of unhybridised nucleic acid molecules and hybridisation-mismatched nucleic acid molecules and qualitatively and/or quantitatively detecting said enriched variable region nucleic acid molecule.

Preferably said neoplastic condition is a malignant condition and even more preferably said mammal is a human.

Reference to “derivatives” and “analogues” should be understood to include reference to fragments, parts, portions, mutants, homologues, mimetics and analogues from natural, synthetic or recombinant sources. The derivatives of said nucleic acid molecules include fragments having particular epitopes or parts of the nucleic acid sequence. For example, the reference molecule may encode only part of the variable region if this is a sufficient marker. Similarly, a part only of the variable region may provide a sufficient marker, such as the DJ region only of an immunoglobulin rearrangement or the junction point only. This definition also includes nucleic acid molecules fused to other proteinaceous or non-proteinaceous molecules. The subject nucleic acid molecules may be fused to tags, for example which facilitate the isolation or detection of said molecules. Analogs contemplated herein include, but are not limited to, modifications to the nucleic acid sequence such as modifications to its chemical makeup or overall conformation. For example, the reference nucleic acid molecule may exhibit a uracil nucleotide for the purpose of providing a enzymatic cleavage target to enable subsequent removal of any unwanted reference molecules which have carried over. This also includes, for example, modification to the manner in which nucleic acid sequences interact with other nucleic acid sequences such as at the level of backbone formation or complementary base pair hybridisation. The biotinylation of a nucleotide or nucleic acid sequence is an example of a derivative as herein defined. Derivatives of nucleic acid sequences may be derived from single or multiple nucleotide substitutions, deletions and/or additions. The term “derivatives” should also be understood to encompass nucleic acid sequences exhibiting any one or more of the activities of a nucleic acid sequence, such as for example, products obtained following natural product screening and also to encompass nucleotide sequences on different backbones such as peptide nucleic acids.

Contacting the reference nucleic acid molecule with the test sample nucleic acid molecule such that interaction is facilitated with any marker molecule present in the test sample may be performed by any suitable method. These methods will be known to those skilled in the art. In this regard, reference to “interaction” should be understood as a reference to any form of interaction such as hybridisation between complementary nucleotide base pairs or some other form of interaction such as the formation of bonds between nucleic acid portions of the subject nucleic acid molecules. The interaction may occur via the formation of bonds such as, but not limited to, covalent bonds, hydrogen bonds, Vanderwaal's forces or any other mechanism of interaction. All references herein to “hybridisation” between two nucleic acid molecules should be understood to encompass any form of interaction between said molecules. In order to facilitate this interaction, it is preferable that both the reference nucleic acid molecule and the nucleic acid molecules of the test sample be rendered partially or fully single stranded for a time and under conditions sufficient for hybridization between a single stranded reference molecule and a single stranded marker molecule to occur. It should be understood that hybridisation may occur between the coding strand of the reference nucleic acid molecule and the strand of the marker nucleic acid molecule which is complementary to it, or the non-coding strand of the reference nucleic acid and the strand of the marker nucleic molecule which is complementary to it.

The phrase “nucleic acid molecule” in the context of the reference molecule should be understood to mean any molecule comprising a sequence of nucleotides, or derivatives thereof, the function of which includes the hybridisation of at least one region of said nucleotide sequence to a marker nucleic acid sequence. Accordingly, the phrase “marker nucleic acid molecule” includes any molecule comprising a sequence of nucleotides or derivatives thereof but which molecule is characteristic of the neoplastic cell, as hereinbefore described, and is therefore the subject of identification via the contacting step. Both the nucleic acid reference molecule and the marker nucleic acid molecule may comprise non nucleic acid components such as tags which facilitate the detection and/or enrichment of these molecules. These tags may be incorporated at any suitable time point during the performance of the subject method.

Without limiting the theory or mode of operation of the present invention in any way, contacting the test sample nucleic acid molecules with the reference nucleic acid molecule under hybridisation conditions may result in the formation of reference:marker homoduplexes, reference:reference homoduplexes or marker:marker homoduplexes. Heteroduplexes will be formed where a reference molecule hybridizes in a mismatched fashion with a non-marker molecule or where marker or non-marker nucleic acid molecules hybridise with nucleic acid molecules of different sequences. The concentration of marker:marker homoduplex formation can be minimised by contacting the test sample with an excess of reference nucleic acid molecules.

Still without limiting the invention in any way, hybridisation of the reference nucleic acid molecule with a marker nucleic acid molecule present in the test sample facilitates the enrichment of the marker nucleic acid molecule in a simple yet specific manner. Specificity is provided by the fact that the marker molecule is hybridised to the reference molecule to form a homoduplex thereby differentiating the marker molecule from other non-marker, heterogeneous nucleic acid molecules which also form part of the test sample.

Reference to “enriching” should be understood as a reference to increasing the ratio of marker nucleic acid molecules relative to the background non-marker nucleic acid molecules contained in the test sample. This can be achieved, for example, by degrading, removing or otherwise reducing the non-marker nucleic acid molecules. In accordance with the method of the present invention, the enrichment step takes the form of decreasing the concentration of non-marker nucleic acid molecules contained in the sample rather than by increasing the concentration of the marker nucleic acid molecules within the test sample by amplifying the marker nucleic acid molecules. Without limiting the theory or mode of operation of the present invention in any way, the inventors have found that by utilising an enrichment step which is predicated on decreasing non-marker nucleic acid molecule concentrations from the test sample, rather than attempting to amplify the marker nucleic acid population, the subject detection method provides a highly sensitive tool which is not compromised by the risk of non-specific amplification occurring. Further, the type of enrichment utilised herein is rendered feasible due to the use of a reference nucleic acid molecule which targets the subject marker nucleic acid molecule. The overall simplicity of this molecular screening method is similarly related to the use of a reference nucleic acid molecule which thereby obviates the need to conduct a sequence analysis of prospective marker nucleic acid molecules in order to facilitate the design of highly specific probes and/or primer molecules.

It should be understood that reference to “enrichment” is not limited to an enrichment step which removes all non-marker nucleic acid molecules from the test sample. Rather, in accordance with the definition provided earlier, it is a reference to decreasing the concentration of non-marker nucleic acid molecules in the test sample. The decrease in concentration may therefore be of varying degrees. The method of the present invention should be understood to extend to conducting one or more sequential enrichment steps in order to improve the purity of the marker nucleic acid molecule population. The decision as to whether one or more enrichment steps are required to be performed can be made by a person skilled in the art on a case by case basis. When neoplastic lymphoid numbers are high, for example during the early stages of treatment, a single, simple enrichment step may be sufficient to detect the marker nucleic acid molecules which would be present in high concentrations. However, where a patient who is in remission is the subject of testing, it may be desirable to perform two or more different enrichment techniques in order to maximise the purity of the subject marker molecule.

Enriching for marker nucleic acid molecules can be performed by any one or more of a number of suitable techniques including, but not limited to:

(i) Incorporating a tag into either the reference nucleic acid molecules or the nucleic acid molecules derived from the test sample. The tag can be used to couple molecules to a solid phase whether by covalent bonds or by non-covalent bonds in order to facilitate removal of unwanted molecules by washing or other means. The tag can also be used to provide a nucleic acid sequence on either the nucleic acid molecules derived from the test sample or the reference nucleic acid which is resistant to enzymatic cleavage. Such tags are known to those skilled in the art and commonly used ones include biotin digoxigen and fluorescein. Another commonly used tag involves a peptide or phosphorothioate backbone which is resistant to enzymes. Solid phases suitable for such use include plastic surfaces, gel matrixes and coated magnetic beads and they may have attached to them capture molecules such as streptavidin or antibody.

(ii) Separating reference:marker homoduplexes from hybridisation-mismatched heteroduplexes based on differential migration of the duplexes through a gel or a size exclusion or affinity or other matrix contained in an apparatus such as a column or capillary.

(iii) Removing hybridization mismatch heteroduplexes, such as those formed between a reference nucleic acid molecule which is partially hybridised to a non-marker nucleic acid molecule, via association of such a heteroduplex with a mismatch repair protein. Removal may be facilitated by any suitable technique such as where the mismatch repair protein is coupled to a solid phase surface such as the surface of a column or plate. In another example, use may be made of the mismatch repair protein differentially altering migration of heteroduplexes through a gel or matrix.

(iv) Removing unmatched single stranded nucleic acid molecules and/or hybridisation-mismatched heteroduplexes utilising chemical or enzymatic cleavage techniques. Preferably, enzymatic techniques are utilised such as digestion of the subject nucleic acid molecules utilising S1 nuclease and/or T4 endonuclease.

(v) Coupling tagged homodimers and heterodimers to a solid phase and removing unhybridised single stranded molecules, heterodimers or homodimers. This removal step may be achieved, for example, by washing and then heating or chemically treating the remaining population of nucleic acid molecules in order to render single stranded any non-marker molecules which have hybridised in a mismatched fashion to the reference nucleic acid molecule. Without limiting the present invention in any way, hybridisation-mismatched non-marker molecules melt at a lower temperature than perfectly hybridised marker molecules. The newly single stranded molecules can then be removed by a washing step.

It should be understood that the subject enrichment step may be achieved by performing any one or more suitable techniques, such as one or more of the techniques outlined in points (i)-(v), above. The subject techniques may be performed in any number of ways including in solution, via a separating gel or column or via attachment of either the reference nucleic acid molecule or the test sample nucleic acid molecule to a solid phase. It will fall within the capability of the person of ordinary skill in the art to determine the most suitable enrichment protocol. Where the enrichment comprises the use of more than one technique, the techniques are preferably performed sequentially.

In addition to applying any one or more of the above detailed enrichment techniques, the driver molecule is preferably removed prior to detecting the marker nucleic acid molecules. This further enriches the marker nucleic acid population. This can be achieved by any one of a number of techniques including:

(i) Rendering the isolated nucleic acid molecule duplexes single stranded and separating out the reference nucleic acid molecule by virtue of a tag incorporated into that molecule. For example, a biotin label or magnetic bead as hereinbefore described.

(ii) Specifically degrading the reference molecule by virtue of a suitable identification tag which has been incorporated into the reference nucleic acid molecule. For example, incorporation of a uracil analogue into the reference molecule DNA permits specific digestion of the reference molecule utilising the enzyme uracil N glycocylase (UNG). Without limiting the present invention in any way, to the extent that reference:reference molecule homoduplexes have formed or that driver:non-marker heteroduplexes have survived enrichment, a step such as this will remove these excess molecules.

(iii) Utilizing an enzyme-resistant tag attached only to the nucleic acid molecule derived from the test sample to enable amplification of these molecules but not the reference molecule. This greatly decreases the relative proportion of the reference molecule.

In a most preferred embodiment, the step of enriching for the marker molecule by reducing concentrations of unhybridised nucleic acid molecules and hybridisation-mismatched nucleic acid molecules further includes the step of reducing the concentration of reference nucleic acid molecules.

According to this most preferred embodiment there is provided a method for monitoring a neoplastic lymphoid condition in a mammal, said method comprising contacting the nucleic acid molecules contained in a test sample derived from said mammal with a nucleic acid driver molecule or derivative or analogue thereof which is complementary to a rearranged TCR or immunoglobulin variable region nucleic acid molecule or derivative or analogue thereof for a time and under conditions sufficient to facilitate the interaction of said driver molecule with said variable region nucleic acid molecule, enriching for said variable region nucleic acid molecule by reducing the concentration of unhybridised nucleic acid molecules, hybridisation-mismatched nucleic acid molecules and nucleic acid driver molecules and qualitatively and/or quantitatively detecting said enriched variable region nucleic acid molecule.

In a most preferred embodiment the enrichment step is performed by removing non-marker nucleic acid molecules which have not hybridised to a tagged driver nucleic acid molecule, enzymatically cleaving mismatched hybridisation nucleic acid molecules and removing driver molecules, for example by the use of UNG.

“Detection” of the marker molecule can be performed by any suitable method known to those skilled in the art. In this regard, reference to “qualitative” detection should be understood as a reference to detecting the presence or absence of a neoplastic lymphoid cell population while “quantitative” detection should be understood as a reference to detecting the levels of neoplastic lymphoid cells present in the subject mammal. Detection techniques which are suitable for use in the method of the present invention include, but are not limited to:

(i) Labelling the enriched marker nucleic acid molecule population with a detection tag which emits a signal or which can be coupled to a detection system which emits a signal and then detecting said signal. This includes, for example, colorimetric detection, fluorescent detection, enzymatic detection or detection of radioactive tags; or

(ii) Amplifying the enriched marker nucleic acid molecule prior to its detection. This may be required, for example, where the copy number of marker nucleic acid is low (for example because a patient is in remission). Since the marker nucleic acid population has been enriched, it is not necessary to synthesize amplification primers specifically directed to the subject marker molecule. Rather, universal primers can be utilised to amplify the subject marker nucleic acid population and the amplified product can be detected by electrophoresis.

(iii) Quantitative detection of the (unknown) marker can be achieved by adding to the initial test sample a known amount of a standard of different sequence, performing initial enrichment using two reference drivers, one for the standard and one for the marker, and determining the relative amounts of enriched standard and unknown marker molecules which are finally obtained. The amount of starting unknown marker can then be calculated.

(iv) 2-dimensional electrophoretic separation, as hereafter described, can be used.

Without limiting the invention in any way, the inventors have demonstrated that the method of the present invention enables detection and quantification of disease in blood down to a level of 10⁻⁵ and in marrow down to a level of 10⁻⁵ to 10⁻⁶ leukaemic cells per total cells in sample. This is a thousand-fold improvement over the currently available techniques of equivalent performance simplicity (refer table 1).

In a related aspect, the present inventors have determined that the marker nucleic acid molecules hereinbefore defined exhibit unique electrophoretic migration patterns due to differences which these molecules exhibit in their size and nucleotide sequence. Accordingly, the present inventors have developed a method for qualitatively and/or quantitatively detecting the presence of a population of marker nucleic acid molecules in a biological sample based on the electrophoretic separation of the marker population from the heterogeneous non-marker nucleic acid molecules contained in a test sample to form an isolated and therefore detectable population. By screening for the presence of individual marker nucleic acid populations, it is possible to determine whether expansion of a clonal population of cells, such as is observed in neoplastic conditions, has occurred.

Accordingly, another aspect of the present invention is directed to a method for detecting and/or quantifying a clonal population of cells in a biological sample said cells being characterised by a marker nucleic acid molecule, which marker nucleic acid molecule is electrophoretically co-migratable within said population of cells, said method comprising electrophoretically separating the nucleic acid molecules contained in said sample, wherein said separation is based on nucleic acid length and sequence, and detecting said separated nucleic acid molecules.

It should be understood that the phrase “being characterised by” is intended to indicate that the subject cells exhibit the defined characteristic but it is not intended as a limitation in respect of what other characteristics the cell might also exhibit. It should also be understood that the subject characteristic is not necessarily uniquely exhibited only by the subject cells although in a preferred embodiment the subject characteristic is one which identifies the cell of interest from the cells of non-interest which are present in the sample.

By “clonal” is meant that the subject population of cells has derived from a common cellular origin. For example, a population of neoplastic cells is derived from a single cell which has undergone transformation. In this regard, a neoplastic cell which undergoes further nuclear rearrangement or mutation to produce a genetically distinct population of neoplastic cells is also a “clonal” population of cells. In another example, a T or B lymphocyte which expands in response to an acute or chronic infection or immune stimulation is also a “clonal” population of cells within the definition provided herewith. Preferably, the clonal population of cells is a population of neoplastic cells and even more preferably neoplastic lymphoid cells.

Accordingly, the present invention more particularly provides a method for detecting and/or quantifying a population of neoplastic lymphoid cells in a biological sample said cells being characterised by a marker nucleic acid molecule, which marker nucleic acid molecule is electrophoretically co-migratable within said population of cells, said method comprising electrophoretically separating nucleic acid molecules contained in said sample, wherein said separation is based on nucleic acid length and sequence, and detecting said separated nucleic acid molecules.

Reference to “neoplastic” and “lymphoid” should be understood to have the same meaning as hereinbefore provided.

Reference to “marker nucleic acid molecule” has the same meaning as hereinbefore provided. To the extent that the subject neoplastic cells are lymphoid cells, the subject marker is preferably the rearranged TCR or immunoglobulin variable region nucleic acid molecule.

According to this preferred embodiment there is provided a method of detecting and/or quantifying a population of neoplastic lymphoid cells in a biological sample said neoplastic cell being characterised by a marker nucleic acid molecule which marker nucleic acid molecule is electrophoretically comigratable within said population of cells, said method comprising electrophoretically separating the nucleic acid molecules contained in said sample, wherein said separation is based on nucleic acid length and sequence and detecting said separated nucleic acid molecules wherein the rearranged TCR or immunoglobulin variable region nucleic acid molecules of said neoplastic lymphoid cells co-migrate.

Reference to a “biological sample” should be understood as a reference to a mammalian derived test sample as hereinbefore defined. To the extent that the sample comprises cellular material, it may be necessary to extract or otherwise isolate or expose the nucleic acid material contained in the sample. As detailed earlier, to the extent that the subject marker nucleic acid molecule is a mRNA molecule, it may be necessary to initially apply a stimulatory signal to the test sample in order to up-regulate production of this transcription product. It may also be desirable to amplify the marker nucleic acid population prior to testing, where specific primers are available, or to amplify the nucleic acid population of the test sample as a whole, utilising universal primers, for the purpose of providing a larger starting population of nucleic acid molecules.

Without limiting the present invention to any one theory or mode of action, marker molecules such as the rearranged TCR or immunoglobulin variable region gene exhibit unique nucleotide sequences which thereby encode unique TCR or immunoglobulin variable regions. The inventors have determined that when a nucleic acid sample containing such molecules is subjected to electrophoretic separation based on separation according to the length of the individual molecules and their nucleotide sequence, the marker molecules co-migrate to a common end point on the electrophoretic gel. The nucleic acid migration pattern which is finally obtained can be visualised on the gel utilising a technique such as autoradiography, fluorography or the visualisation of a molecule (such as a fluorescent tag or antigen) which has been incorporated into the nucleic acid molecules of the test sample prior to, during or after electrophoretic separation.

Since the numerous populations of nucleic acid molecules comprising a test sample will have each migrated to a unique length/sequence electrophoretic position, and since it is possible to either relatively or absolutely determine the quantity of nucleic acid material present in any given position, it can be quickly and simply determined whether a population of neoplastic lymphoid cells was present in the tested biological sample due to the presence of a higher concentration of the subject marker nucleic acid molecule relative to the levels of the non-marker nucleic acid molecule population.

In this regard, reference to the marker nucleic acid molecule being “co-migratable within said population of cells” is a reference to the subject marker nucleic acid molecules, to the extent that they are derived from cells which derive from the same clonal population, electrophoretically migrating to the same end point when the electrophoretic separation is based on the parameters of length (ie total number of base pairs) and sequence (ie the actual nucleotide sequence of the subject nucleic acid molecules). Without limiting the present invention to any one theory or mode of action, the present invention is based on using the sequence differences which exist between the marker molecule of a neoplastic population of cells and a non-neoplastic heterogeneous population of cells to electrophorectically separate a clonal population of nucleic acid molecules.

Electrophoretic separation of the molecules based on length and sequence can be performed utilising any suitable technique. In a preferred embodiment, the technique is two dimensional denaturing gradient gel electrophoresis wherein the test nucleic acid molecule population is first separated according to length, the molecules are then run through a urea gradient gel. Due to the fact that urea's differential interference with a nucleic acid molecule's H-bonding is sequence dependent, this provides one example of a suitable method for separating nucleic acid molecules based on nucleotide sequence differences. Temperature can also be used as a denaturant. Constant or gradient denaturing conditions can be used.

Accordingly, the present invention more particularly provides a method for detecting and/or quantifying a population of neoplastic lymphoid cells in a biological sample said neoplastic cell being characterised by a marker nucleic acid molecule, which marker nucleic acid molecule is electrophoretically co-migratable within said population of cells, said method comprising electrophoretically separating the nucleic acid molecules contained in said sample, wherein said separation is two dimensional denaturing gradient gel electrophoresis and is based on nucleic acid length and sequence, and detecting said separated nucleic acid molecules.

Preferably, said neoplastic cells are malignant.

Without limiting the present invention in any way, the inventor has determined that 2 dimensional electrophoresis increases the sensitivity of detection of marker nucleic acid molecules in a population of non-marker nucleic acid molecules by a factor of 10-100.

The method of this aspect of the present invention provides a simple yet sensitive method of detecting the presence of clonal populations of cells in a mammal. The method is particularly useful for screening for large clonal populations as evidenced by higher concentrations of nucleic acid molecules which have co-migrated to a particular end point relative to the concentrations of nucleic acid molecules detected comparatively, or relative to a quantitative standard, at other end point positions. Although the method of the present invention could be used as a diagnostic tool, it is particularly useful for monitoring neoplastic conditions in terms of detecting modulation in the size of a population of neoplastic cells in a mammal or for detecting the incidence of clonal evolution of neoplastic cells.

Without limiting the present invention in any way neoplastic lymphoid cells can undergo further rearrangement of the variable region genes since not all unused variable region segments are necessarily spliced out at the time of initial rearrangement of the germline genes during the cell's early differentiative stages. For example, leukaemia patients who relapse two to five years after entering remission sometimes exhibit a new TCR or immunoglobulin rearrangement. This is usually due to clonal evolution of the original neoplastic cells before or after diagnosis. Using the method of the present invention, it is possible to identify a clonal population, such as a new clonal population, in a sensitive manner without the need for nucleotide sequence information. When this method is used as a monitoring tool over a period of time, changes in the size or source of populations of cells can be tracked. For example, with certain neoplastic conditions evidence of the presence of neoplastic cells in the bone marrow would be linked to a poor prognosis. In another example, it is useful as a predictive tool during treatment of a neoplastic condition. Specifically, following 5 weeks of treatment, a patient who still exhibits high levels of neoplastic cells will generally have a poorer prognosis than a patient whose level of neoplastic cells has decreased to low levels.

The method of this aspect of the present invention can be used in addition to or instead of the method of testing described in the first aspect of the present invention. Without limiting the present invention in any way, the first aspect of the present invention is likely to provide a more sensitive result than the latter since neoplastic cells are detected utilising a reference molecule which detects a marker molecule therefore obviating the need for the neoplastic cells to be present in higher numbers relative to other normal clonal populations of cells in order to achieve a positive result. Although both methods obviate the need for marker nucleic acid sequence information relating to the neoplastic cell, the latter aspect of the present invention further obviates the need for reference molecule. This is likely to be of particular use where clonal evolution is the subject of detection and reference nucleic acid molecules corresponding to the new clone have not yet been obtained. In this respect, the latter aspect of the present invention effectively provides both a monitoring and a diagnostic tool.

Accordingly, in a related aspect the present invention provides a method for detecting and/or quantifying neoplastic cells in a mammal, said neoplastic cells being characterised by a marker nucleic acid molecule which marker nucleic acid molecule is electrophoretically co-migratable within said population of cells, said method comprising electrophoretically separating nucleic acid molecules contained in a sample derived from said mammal, wherein said separation is based on nucleic acid length and sequence, and detecting said separated nucleic acid molecules.

Preferably, said neoplastic condition is the clonal evolution of a neoplastic cell. Most preferably, said detection is diagnosis.

Still more preferable, said electrophoretic separation is two dimensional denaturing gradient gel electrophoresis.

In another aspect there is provided a method for detecting and/or quantifying multiple non-neoplastic lymphoid cells in a biological sample, said non-neoplastic cells being characterised by a marker nucleic acid molecule, which marker nucleic acid molecule is electrophoretically co-migratable within said population of cell, said method comprising electrophoretically separating the nucleic acid molecules contained in said sample, wherein said separation is based on nucleic acid length and sequence, and detecting said separated nucleic acid molecules.

Preferably said separation is two dimensional denaturing gradient gel eletrophoresis.

Further features of the present invention are more fulls described in the following non-limiting examples.

### EXAMPLE 1

The leukaemic rearrangement, obtained at diagnosis, termed “driver” is added in excess to the test DNA. This driver is modified in one of several ways in order to enable its subsequent removal later during the process. The DNA is then denatured and allowed to reanneal. As a result, the driver associates either with leukaemic test molecules or with normal test molecules. Association with leukaemic test molecules will produce a perfect match, whereas association with normal heterogeneous rearrangements, which have a different sequence, will produce mismatches. The mismatches can then be removed by physical means, either based upon differential migration through columns or down capillaries, or by association with mismatch repair proteins which are physically attached to a surface such as a column or a plastic surface. The driver is then removed and the enriched leukaemic molecules, separated from the “noise” of the normal molecules can then be detected and quantified (see Example 2).

### EXAMPLE 2

The initial procedure is the same as in Example 1. Leukaemic driver is added in excess to the test DNA and denaturation is performed followed by reannealing. Again the driver associates either with leukaemic test molecules or with normal test molecules. Mismatches are now removed chemically or enzymatically, as the result of chemical processes or enzymes which recognise mismatches. As a result, the cleaved molecules will not then amplify in a subsequent polymerase chain reaction. The leukaemic driver molecules are removed by various enzymes such as uracil-N-glycosylase, nucleases or restriction enzymes. Quantification is performed by adding a known amount of a standard clonal DNA to the original test DNA. Standard driver is also used to protect the standard DNA. Following removal of the heterogeneous normal rearrangements and the driver DNAs, a final PCR is performed using fluorochrome labelled primers. The relative amounts of standard and marker are determined using an instrument such as the gene sequencer and appropriate software and measuring fluorescence during gel electrophoresis. It has been demonstrated that this method by itself enables detection and quantification of disease in blood down to a level of 10⁻⁵ and in marrow down to a level of 10⁻⁵-10⁻⁶ leukaemic cells per total cells in sample. This is a thousand-fold improvement over the current simple methods which are applicable to the majority of patients referred to in the Table 1.

### EXAMPLE 3

This is based upon the principle of using sequence differences between the leukaemic and heterogeneous normal rearrangements. Sequence differences can be detected by denaturing gradient gel electrophoresis. The leukaemic and normal gene rearrangements in the test material will be amplified by polymerase chain reaction using primers with a GC clamp and will then be subjected to two dimensional gradient gel electrophoresis. Target rearrangements and any reference rearrangements will migrate to more specific points in the two dimensional gel. In order to obtain sensitivity, the DNA in the gel will be transferred to a membrane and will then be detected by radioactive or chemical means. Although two dimensional denaturing gradient gel electrophoresis is not a new method and one dimensional DGGE has been used for detecting clonal lymphocyte rearrangements, the use of two dimension denaturing gradient gel electrophoresis to detect malignant lymphoid clones is a new use for the product. However, under some circumstances, simple methods for detection of the PCR products may not be sufficiently sensitive. If so, more sensitive methods, such as probing with labelled probes or the use of labelled primers with sensitive secondary detection will be required. Quantitation will be achieved by the use of a reference monoclonal DNA. When this method is used to study the material from Examples 1 and/or 2 the PCR will use primers with a GC clamp rather than the primers mentioned in Example 2 above.

### EXAMPLE 4

### Rapid Methods Protocol

1. Make Driver Single Stranded Using λ Exonuclease: Mix Driver DNA, λ Exonuclease, 10× buffer and H₂O, Incubate

The driver is from leukaemic DNA at diagnosis. It is modified by incorporating uracil so that any trace of driver can be eliminated towards the end of the method.

2. Denature and Anneal Driver and Marker to Form Heteroduplexes and Homoduplexes

The driver is in excess so that all of the marker strands will form duplexes with a driver strand. There will be some excess single stranded driver molecules also present.

3. Treat with S1 Nuclease: Add S1 Enzyme and 10×S1 Buffer, Incubate

The S1 nuclease digests heteroduplexes in which there is a substantial degree of mismatch. It also removes excess single stranded driver.

4. Bind Samples to Prepared Streptavidin Coated Beads in Binding and Wash Buffer (B&W), Incubate on Rotating Wheel, Wash ×3 with B&W, ×1 H₂O

The marker has been prepared using a biotin labelled primer. Therefore, heteroduplexes and homoduplexes, in which a marker molecule forms one strand, will bind to the solid phase owing to binding to streptavidin.

5. Treat with T4 Endonuclease: Add T4 Enzyme, T4 Phosphate Buffer, Incubate, Wash ×3 with B&W, ×1 H₂O

This enzyme digests heteroduplexes.

6. Treat with Cel I Endonuclease: Add Cel I Enzyme. 10×H Buffer, Klenow and H₂O, Incubate, Wash ×3 with B&W, ×1 H₂O, ×3 H₂O at Higher Temp.

This enzyme digests heteroduplexes.

7. Treat with S1 Nuclease: Add S1 Enzyme, 10×S1 Buffer and H₂O, Incubate, Wash ×3 with B&W, ×1 H₂O

This enzyme digests heteroduplexes.

8. Treat with λ Exonuclease: Add λ Exonuclease, 10×λ Buffer and H₂O, Incubate, Wash ×3 with B&W, ×1 H₂O

The exonuclease digests any residual driver molecules.

9. Wash ×1 with Sodium Hydroxide, ×3 B&W, ×3 H₂O at Higher Temp.

This step aims to remove the second strand of any duplexes bound to the beads.

10. Treat with Uracil DNA Glycosylase (UNG): Add UNG Enzyme, UNG Buffer and H₂O, Incubate

Treatment of UNG aims to digest any persisting driver molecules, which will have incorporated Uracil.

11. Perform PCR on a Fraction of the Streptavidin Coated Beads and Run PCR Products on Acrylamide Gel

At the final stage, the process should have resulted in single marker DNA strands Which have survived the procedure as they are a perfect match to the driver molecules. i.e. they have the leukaemic sequence.

Quantification is achieved by performing the same process with a reference DNA molecule which has its own reference driver. AT the beginning of the process, various amounts of the reference molecule are mixed with marker molecules and taken through the process. At the end of the process, the final PCR is performed with fluorescein labelled primers and the relative amounts of the leukaemic marker and the reference are determined by quantification using DNA fragment analysis.

Those skilled in the art will appreciate that the invention described herein is susceptible to variations and modifications other than those specifically described. It is to be understood that he invention includes all such variations and modifications. The invention also includes all of the steps, features, compositions and compounds referred to or indicated in this specification individually or collectively, and any and all combination of any two or more of said steps of features.

TABLE 1
Current methods for detection and for quantifiction of neoplastic lymphoid cells in a population

| Marker | Method | Applicability | Complexity | Sensitivity |
|---|---|---|---|---|
| 1 surface/intracellular antigens | flow cytometry | 50%-70% | intermediate | intermediate (10⁻¹) |
| 2 molecular markers | | | | |
| (a) translocation | PCR | minority | simple | high (10⁻⁶) |
| (b) gene rearrangement | | | | |
| detection by monoclonality | PCR | ˜90% | simple | low (10⁻³) |
| specific probing | PCR | 90% | high | intermediate |
| specific primer generation | PCR | 90% | high | high (10⁻⁶) |

1. A method for monitoring a lymphoid neoplastic condition in a mammal, said method comprising contacting the nucleic acid molecules contained in a sample derived from said mammal with a nucleic acid reference molecule or derivative or analogue thereof which is complementary to a marker nucleic acid molecule or analogue thereof, which marker molecule is characteristic of the neoplastic cells, for a time and under conditions sufficient to facilitate the interaction of said reference molecule with said marker molecule, enriching for said marker molecule by reducing the concentration of un-hybridised nucleic acid molecules and hybridisation-mismatched nucleic acid molecules and qualitatively and/or quantitatively detecting said enriched marker nucleic acid molecules.

2. The method according to claim 1 wherein said reference nucleic acid molecule is a driver nucleic acid molecule.

3. The method according to claim 1 or 2 wherein the step of enriching for the marker molecule by reducing concentrations of un-hybridised nucleic acid molecules and hybridisation-mismatched nucleic acid molecules further includes the step of reducing the concentration of reference nucleic acid molecules.

4. The method according to claim 3 wherein the enrichment step is performed by removing non-marker nucleic acid molecules which have not hybridised to a tagged driver nucleic acid molecule, enzymatically cleaving any mismatched hybridisation nucleic acid molecules, and removing driver molecules.

5. The method according to claim 3 wherein the enrichment step is performed by separating reference marker homoduplexes from hybridisation and mismatched heteroduplexes based on differential migration of the duplexes through a gel or a size exclusion or affinity or other matrix.

6. The method according to claim 5 wherein said matrix is contained in a column or capillary.

7. The method according to claim 4 wherein said driver molecules are removed by the use of UNG.

8. The method according to any one of claims 1-7 wherein said marker is a rearranged TCR or immunoglobulin variable region nucleic acid molecule or derivative or analogue thereof.

9. The method according to claim 8 wherein said variable region nucleic acid molecule is the genomic form of the rearranged variable region gene segment.

10. The method according to claim 9 wherein said lymphoid neoplastic condition is a lymphoid malignant condition.

11. The method according to claim 10 wherein said monitoring is monitoring of the minimal residual disease condition.

12. A method for detecting and/or quantifying a clonal population of cells in a biological sample said cells being characterised by a marker nucleic acid molecule, which marker nucleic acid molecule is electrophoretically co-migratable within said population of cells, said method comprising electrophoretically separating the nucleic acid molecules contained in said sample, wherein said separation is based on nucleic acid length and sequence, and detecting said separated nucleic acid molecules.

13. The method according to claim 12 wherein said clonal population of cells is a population of neoplastic cells.

14. The method according to claim 13 wherein said neoplastic cells are malignant.

15. The method according to claim 13 or 14 wherein said neoplastic cells are neoplastic lymphoid cells.

16. The method according to claim 15 wherein said marker is a rearranged TCR or immunoglobulin variable region nucleic acid molecule or derivative or analogue thereof.

17. The method according to claim 16 wherein said variable region nucleic acid molecule is the genomic form of the rearranged variable region gene segment.

18. The method according to any one of claims 12-17 wherein said separation is two dimensional denaturing gradient gel electrophoresis.

19. The method according to anyone of claims 12-18 wherein the subject neoplastic condition is the clonal evolution of a neoplastic cell.

20. A method for detecting and/or quantifying multiple non-neoplastic lymphoid cells in a biological sample, said non-neoplastic cells being characterised by a marker nucleic acid molecule, which marker nucleic acid molecule is electrophoretically co-migratable within said population of cells, said method comprising electrophoretically separating the nucleic acid molecules contained in said sample, wherein said separation is based on nucleic acid length and sequence, and detecting said separated nucleic acid molecules.

21. The method according to claim 20 wherein said separation is two dimensional denaturing gradient gel electrophoresis.

22. The method according to any one of claims 1-21 substantially as hereinbefore described with reference to the Figures and/or Examples.
